(19) United States
(12) Patent Application Publication
(10) Pub. No.: US 2023/0363964 A1
(43) Pub. Date: Nov. 16, 2023

(54) PET BED WITH VITAL SIGN MONITORING

(71) Applicant: Brisby, Inc., Newton, MA (US)

(72) Inventor: Jamie Gordon Nichol, Carlisle, MA (US)

(21) Appl. No.: 18/196,034

(22) Filed: May 11, 2023

Related U.S. Application Data
(60) Provisional application No. 63/340,649, filed on May 11, 2022.

(51) Int. Cl. A61G 7/05; A61B 5/0205

(52) U.S. Cl. CPC A61G 7/05; A61B 5/0205; A61B 2503/40; A61B 2562/0219; A61B 2562/0252

(57) ABSTRACT

Systems and method for monitoring vital signs of a patient include a platform having a top surface upon which a patient may rest and a bottom surface; a vibration sensor coupled to the platform such that vibrations caused by the resting patient are coupled to the vibration sensor; a set of load cells coupled to the bottom surface, wherein each load cell is isolated from environmental vibrations in a manner that eliminates or dampens environmental vibrations that otherwise might be conducted by the vibration sensor; and at least one processor configured to process the vibration signals and the strain signals and to derive at least one vital signal parameter of the patient based on the processed vibration signals and the processed strain signals including at least one of a patient heart performance parameter or a patient respiration performance parameter.

### CROSS-REFERENCE TO RELATED APPLICATION(S)

This patent application claims the benefit of U.S. Provisional Patent Application No. 63/340,649 entitled PET BED WITH VITAL SIGN MONITORING filed May 11, 2022, which is hereby incorporated herein by reference in its entirety.

The subject matter of this patent application may be related to the subject matter of U.S. patent application Ser. No. 17/412,996 entitled ANIMAL CARE AND MONITORING PLATFORM filed Aug. 26, 2021 and published as U.S. Patent Application Publication No. US 2022/0061765 on Mar. 3, 2022, which claims the benefit of U.S. Provisional Patent Application No. 63/071,181 entitled ANIMAL CARE AND MONITORING PLATFORM filed Aug. 27, 2020, each of which is hereby incorporated herein by reference in its entirety.

### FIELD OF THE INVENTION

The invention generally relates to a pet bed or platform for monitoring heart rate, respiration rate, and other aspects of a pet or other animal.

### BACKGROUND OF THE INVENTION

Vital sign monitoring such as for monitoring a patient heart parameter or a patient respiration parameter often involves connecting probes to the patient in one form or another.

### SUMMARY OF VARIOUS EMBODIMENTS

In accordance with one embodiment of the invention, a system for monitoring vital signs of a patient includes a platform having a top surface upon which a patient may rest and a bottom surface; a vibration sensor coupled to the platform such that vibrations caused by the resting patient are coupled to the vibration sensor, wherein the vibration sensor is configured to provide vibration signals based on the vibrations caused by the resting patient; a set of load cells coupled to the bottom surface and configured to provide strain signals, wherein each load cell is isolated from environmental vibrations in a manner that eliminates or dampens environmental vibrations that otherwise might be conducted by the vibration sensor; and at least one processor configured to process the vibration signals and the strain signals and to derive at least one vital sign parameter of the patient based on the processed vibration signals and the processed strain signals including at least one of a patient heart performance parameter or a patient respiration performance parameter.

In various alternative embodiments, the vibration sensor may include a geophone or a piezoelectric element. The set of load cells may include a plurality of load cells and may be isolated from environmental vibrations by a set of isolation pads (e.g., elastic material pads) underlying the load cells. The set of load cells may be configured such that there is a load cell in every load path carrying weight of the resting patient.

In still other embodiments, the at least one processor may include analog conditioning circuitry configured to amplify and filter the vibration signals and the strain signals and digital conversion circuitry configured to digitize the amplified and filtered vibration and strain signals.

The analog conditioning circuitry may be configured to filter the vibration signals to isolate frequencies of interest, e.g., by including a high-pass filter (e.g., a Sallen-Key topology fourth-order Chebyshev high-pass filter having a 3 dB point of 10 Hz or lower), a low-pass filter (e.g., a Sallen-Key topology fourth-order Chebyshev low-pass filter having a 3 dB point of no less than 20 Hz), and/or a bandpass filter (e.g., a bandpass filter with a bandwidth of at least 10 Hz centered at 15 Hz) to filter the vibration signals. The analog conditioning circuitry may be configured to amplify the filtered vibration signals by a factor of at least 200. The analog conditioning circuitry may be configured to sum the strain signals from the set of load cells (e.g., using a resistive Wheatstone bridge), amplify the summed strain signals (e.g., using a three op-amp instrumentation amplifier circuit), and filter the summed and amplified strain signals (e.g., using a low-pass filter using an RC circuit with a characteristic frequency of approximately 1 Hz). The analog conditioning circuitry may be configured to amplify the summed strain signals by a factor of at least 1000.

The digital conversion circuitry may include at least one analog-to-digital converter and may be configured to sample the amplified and filtered vibration signals at a frequency of greater than 100 Hz and/or to sample the amplified and filtered strain signals at a frequency of greater than 2 Hz.

The at least one processor may further include vital sign monitoring circuitry configured to derive the at least one vital sign parameter from the digitized vibration and strain signals. For example, the vital sign monitoring system may be configured to identify samples of digitized vibration and strain signals relating to the at least one vital sign parameter and derive the at least one vital sign parameter from the identified samples.

The various components may be configured or packaged in various form factors. For example, the platform, the vibration sensor, the set of load cells, the analog conditioning circuitry, the digital conversion circuitry, and the vital sign monitoring circuitry may be integrated in a single device. Alternatively, the platform, the vibration sensor, the set of load cells, the analog conditioning circuitry, and the digital conversion circuitry may be integrated into a first device, and the vital sign monitoring circuitry may be remote from the first device and may receive the digitized vibration and strain signals from the first device via a communication system.

Additional embodiments may be disclosed and claimed.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 is a schematic diagram showing a top perspective view of a pet bed or platform in accordance with certain embodiments.

FIG. 2 is a schematic diagram showing a bottom perspective view of the pet bed or platform of FIG. 1.

FIG. 3 is a schematic diagram of a pet monitoring system including the pet bed or platform of FIGS. 1 and 2 and a signal processing system.

It should be noted that the foregoing figures and the elements depicted therein are not necessarily drawn to consistent scale or to any scale. Unless the context otherwise suggests, like elements are indicated by like numerals. The drawings are primarily for illustrative purposes and are not intended to limit the scope of the inventive subject matter described herein.

### DESCRIPTION OF ILLUSTRATIVE EMBODIMENTS

Definitions. As used in this description and the accompanying claims, the following terms shall have the meanings indicated, unless the context otherwise requires.

A “set” includes one or more members, even if the set description is presented in the plural (e.g., a set of Xs can include one or more X).

Certain embodiments allow for monitoring vital signs of a patient in a non-invasive manner, particularly when the patient is merely resting on a platform.

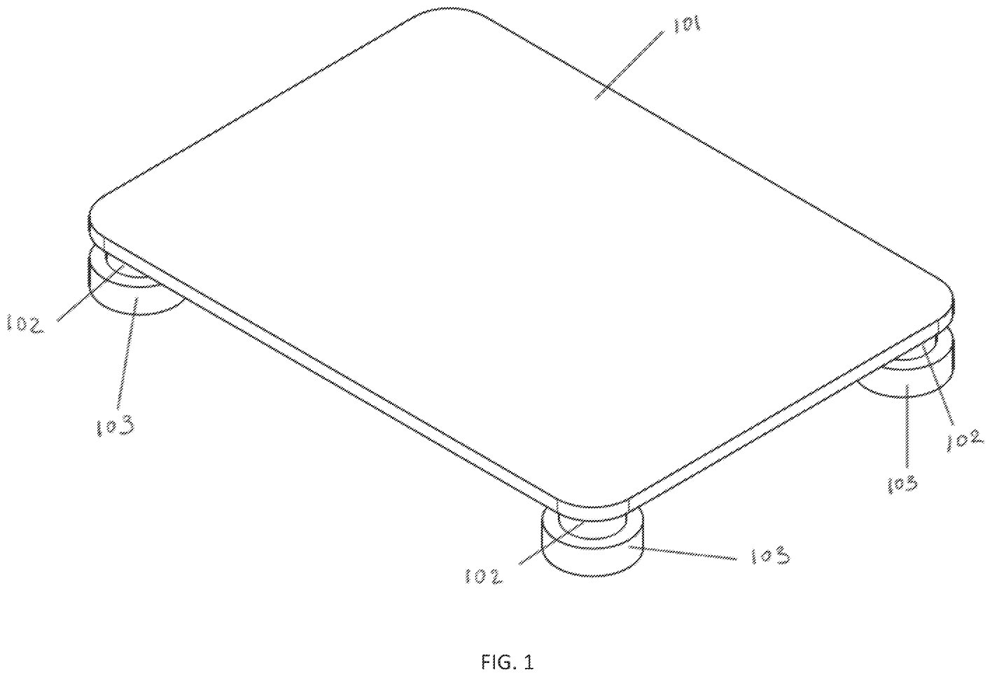

FIG. 1 is a schematic diagram showing a top perspective view of a pet bed 100 in accordance with certain embodiments.

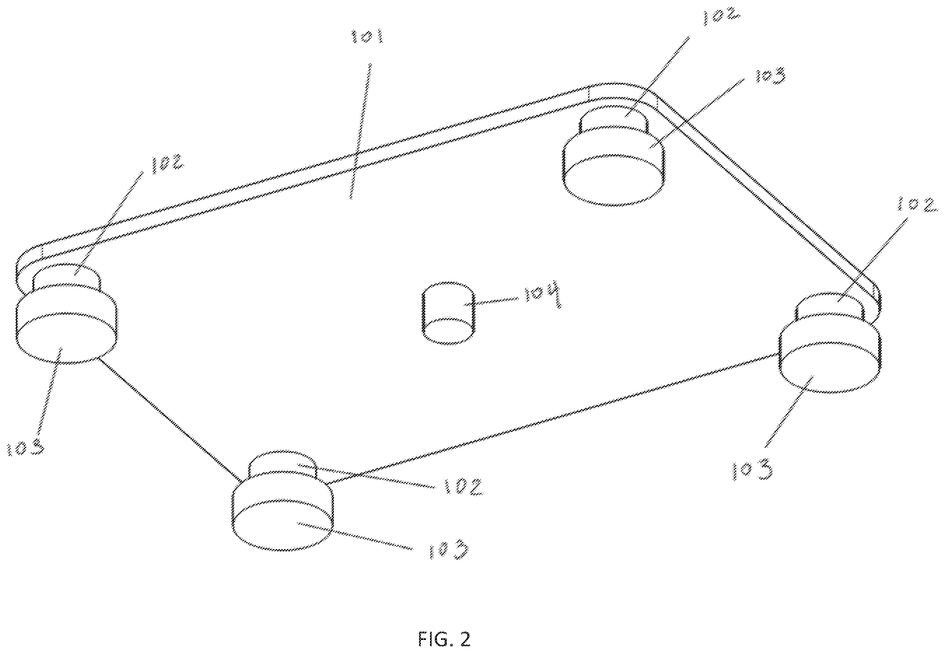

FIG. 2 is a schematic diagram showing a bottom perspective view of the pet bed 100 of FIG. 1.

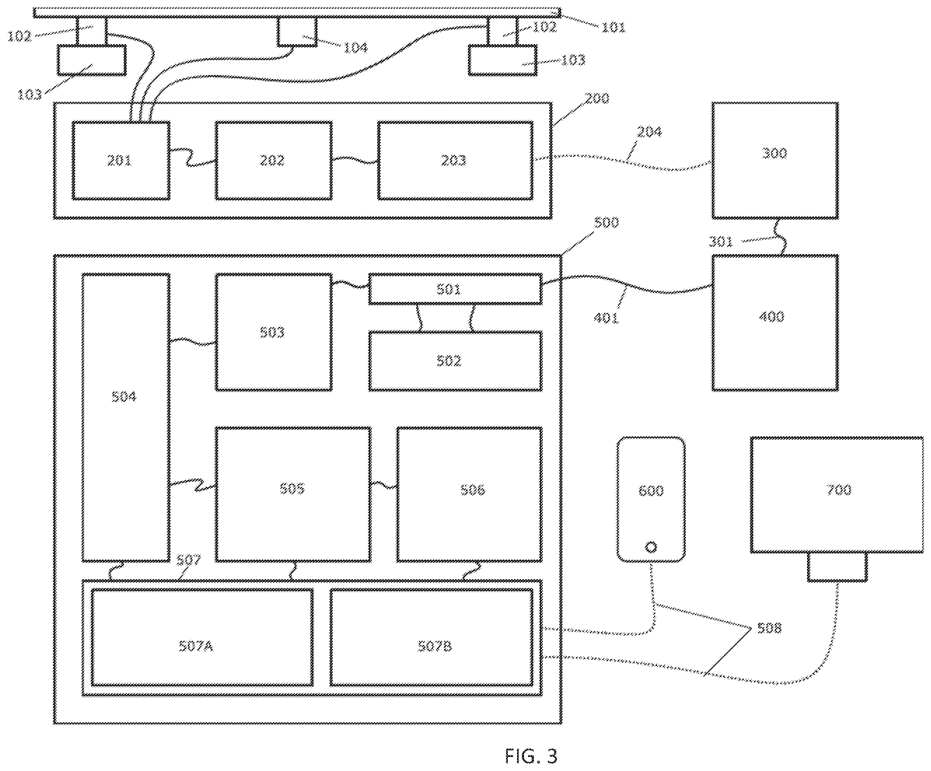

FIG. 3 is a schematic diagram of a pet monitoring system including the pet bed or platform 100 of FIGS. 1 and 2 and a signal processing system with components that may be part of the pet bed or platform, may be located remotely (e.g., in the “cloud” or server system), or distributed between the pet bed and remote locations.

One exemplary pet bed and signal processing system is described below, although it will be understood that the described concepts can be implemented in other ways without departing from the scope of the invention.

It should be noted that the term “pet” is used herein in a non-limiting way and include such things as a pet or other animal, including humans, at any of various stages of life (e.g., for monitoring an infant, toddler, medical patient, convalescent or nursing patent, etc.). It also should be noted that the term “pet bed” is used herein in a non-limiting way and can include such things as a platform that can be placed on or in a separate pet bed or integrated in a scale, cage, or other device in or on which the subject can rest.

The pet bed (100) includes a platform (101) upon which a pet may rest. Connected to the platform is a vibration sensor 104, such as a geophone or piezoelectric element, that connects to platform (101) in a way that allows vibrations caused by the resting pet to be coupled to the vibration sensor (104). Under this platform are arranged a series of load cells (102) such that there is a load cell in every load path carrying the weight of the resting pet. Under each load cell (102) there is an isolation pad (103), e.g., a pad of elastic material (103), which, together with the mass of the pet and bed, acts to isolate the platform from environmental vibrations, e.g., eliminate or dampen environmental vibrations that otherwise might be conducted by the vibration sensor 104.

The signals from this vibration sensor (104), due at least in part to the vibration isolation provided by the elastic pad (103), are indicative of pet movement, which can include such things as heart beats and chest movements due to respirations. These vibrations are converted to an electrical signal by the vibration sensor. This vibration signal together with strain signals from the load cells (102) are conducted to a processing module (200).

These signals are processed first by the analog conditioning circuitry (201). In this embodiment, the signal conditioning circuitry 201 amplifies the vibration signal by a factor of at least 200 and amplifies the strain signals by a factor of at least 1000, and isolates frequencies of interest in the vibration and strain signals.

This circuitry (201) isolates frequencies of interest present in the vibration signals. The high-pass filter for these frequencies can be provided by the inherent dynamics of the vibration sensor (104), e.g., geophone, and/or by analog circuitry in (201), which might include such a Sallen-Key topology fourth-order Chebyshev high-pass filter. In this exemplary embodiment, a high-pass filter would have a 3 dB point of 10 Hz or lower. The circuitry also can include a low-pass filter to further isolate frequencies of interest. Such a filter might be implemented as part of the analog signal conditioning circuitry (201), e.g., in the form of a Sallen-Key topology fourth-order Chebyshev low-pass filter. In this exemplary embodiment, the low-pass filter would have a 3 dB point of no less than 20 Hz. Together, this high-pass and low-pass filter arrangement provides a bandpass filter with a bandwidth of at least 10 Hz centered at 15 Hz. Among other things, this filtering improves noise immunity and prevents aliasing of these analog signals as they are converted to digital signals by an analog-to-digital converter (202).

The analog signal conditioning circuitry (201) also provides summing and low-pass filtering of the load-cell signals. Summing is accomplished by connecting each of the four load cell transducers together in a resistive Wheatstone bridge. The combined signal from this bridge is amplified. This amplification might be accomplished using a classic three op-amp instrumentation amplifier circuit. This signal would be further low-pass filtered using an RC circuit with a characteristic frequency of approximately 1 Hz.

The processed analog signals are converted to digital signals using an analog-to-digital converter (203). This analog-to-digital converter would sample the amplified, bandwidth-limited vibration signal at a frequency of greater than 100 Hz. The load cell signal would be converted to a digital signal using a frequency greater than 2 Hz. These digital signals are then transferred to a WiFi-enabled microcontroller (203). This microcontroller identifies conditions likely to provide heart-and-respiration-rate-disclosing vibrations. Generally speaking, necessary conditions for a heart-and-respiration-rate-disclosing vibration signal include: A pet would need to be present on the platform (101), e.g., as recognized by load cell (102) signals or weight measurements. The pet would ideally be at rest—a few seconds of quiescence generally are sufficient—with quiescence established by monitoring the peak excursions of the vibration signal. A signal within preset limits is understood to recognize quiescence and qualify data as meeting the necessary conditions.

Once these conditions are met, several seconds of vibration data (e.g., 5 seconds or more) are captured together with a single load cell (102) measurement. This combined data is transmitted via a connection to the local area network (204) to a local internet router (300) together with a unique device identifier. This router relays data to the internet (400) via a WAN connection (301). The various intervening switching layers deliver this data to a remote server array (500) via an internet connection (401).

Upon arrival, the data is queued for processing. This queue (501) allows data processing to proceed in an orderly manner without loss of data. Data is pulled from the stack (501) and processed to determine data quality by data triage (502). This data triage (502) determines whether the data will be kept or discarded. Data of sufficient quality, e.g., data likely to contain heart-rate information based on the presence of periodic vibration events in the range of anticipated heart rates (30 bpm to 200 bpm), is returned to the queue (501) flagged as viable. An additional processing system (503) calculates the heart rate, respiration rate and weight from the received data. The heart rate, respiration rate, and weight are associated with a specific animal using the unique device identifier and stored in a database (504). Specific information about the specific pet (age, gender, breed, species) is stored in a separate database (506) together with statistical data about similar pets. The data in the measurement database (504) together with the pet-specific and general data (506) are further analyzed such as using statistical and machine learning techniques (505) to identify trends or other markers which might be worth bringing to the attention of a healthcare provider. This information also can be used to gain addition insight on specific pet categories, e.g., typical heart and breathing rates of different breeds. This information is organized and made available to end users via a user interface (507). This interface can be accessed via API (507A) or GUI (507B). This user interface can be made available via the internet (508) to mobile devices (600) (where the GUI would be created by an on-device application) or computers (700), e.g., where the user interface would be provided by (507) as rendered by a web browser.

It should be noted that embodiments may derive not only heart rate and/or respiration rate from the various signals but in some embodiments also other patient heart performance parameters and patient respiration performance parameters such as, for example and without limitation, heartbeat magnitude, heartbeat anomalies (e.g., heart arrhythmia/irregularity, palpitations, fibrillations, flutters, cessation, etc.), respiration magnitude, and/or respiration anomalies (e.g., irregularity, apnea, cessation, etc.). For example, the system may monitor the time between heartbeats or respirations to detect irregularities or may monitor the magnitude of the vibration signals to infer magnitude of the vital sign parameter.

Various embodiments of the invention may be implemented at least in part in any conventional computer programming language. For example, some embodiments may be implemented in a procedural programming language (e.g., “C”), or in an object-oriented programming language (e.g., “C++”). Other embodiments of the invention may be implemented as a pre-configured, stand-alone hardware element and/or as preprogrammed hardware elements (e.g., application specific integrated circuits, FPGAs, and digital signal processors), or other related components.

In alternative embodiments, the disclosed apparatus and methods (e.g., as in any flow charts or logic flows described above) may be implemented as a computer program product for use with a computer system. Such implementation may include a series of computer instructions fixed on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as a tangible, non-transitory semiconductor, magnetic, optical or other memory device, and may be transmitted using any communications technology, such as optical, infrared, RF/microwave, or other transmission technologies over any appropriate medium, e.g., wired (e.g., wire, coaxial cable, fiber optic cable, etc.) or wireless (e.g., through air or space).

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

Computer program logic implementing all or part of the functionality previously described herein may be executed at different times on a single processor (e.g., concurrently) or may be executed at the same or different times on multiple processors and may run under a single operating system process/thread or under different operating system processes/threads. Thus, the term “computer process” refers generally to the execution of a set of computer program instructions regardless of whether different computer processes are executed on the same or different processors and regardless of whether different computer processes run under the same operating system process/thread or different operating system processes/threads. Software systems may be implemented using various architectures such as a monolithic architecture or a microservices architecture.

Importantly, it should be noted that embodiments of the present invention may employ conventional components such as conventional computers (e.g., off-the-shelf PCs, mainframes, microprocessors), conventional programmable logic devices (e.g., off-the shelf FPGAs or PLDs), or conventional hardware components (e.g., off-the-shelf ASICs or discrete hardware components) which, when programmed or configured to perform the non-conventional methods described herein, produce non-conventional devices or systems. Thus, there is nothing conventional about the inventions described herein because even when embodiments are implemented using conventional components, the resulting devices and systems (e.g., the signal processing system described above) are necessarily non-conventional because, absent special programming or configuration, the conventional components do not inherently perform the described non-conventional functions.

The activities described and claimed herein provide technological solutions to problems that arise squarely in the realm of technology. These solutions as a whole are not well-understood, routine, or conventional and in any case provide practical applications that transform and improve computers and computer routing systems.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Various inventive concepts may be embodied as one or more methods, of which examples have been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein in the specification and in the claims, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Although the above discussion discloses various exemplary embodiments of the invention, it should be apparent that those skilled in the art can make various modifications that will achieve some of the advantages of the invention without departing from the true scope of the invention. Any references to the “invention” are intended to refer to exemplary embodiments of the invention and should not be construed to refer to all embodiments of the invention unless the context otherwise requires. The described embodiments are to be considered in all respects only as illustrative and not restrictive.

What is claimed is:

1. A system for monitoring vital signs of a patient, the system comprising:
a platform having a top surface upon which a patient may rest and a bottom surface;
a vibration sensor coupled to the platform such that vibrations caused by the resting patient are coupled to the vibration sensor, wherein the vibration sensor is configured to provide vibration signals based on the vibrations caused by the resting patient;
a set of load cells coupled to the bottom surface and configured to provide strain signals, wherein each load cell is isolated from environmental vibrations in a manner that eliminates or dampens environmental vibrations that otherwise might be conducted by the vibration sensor; and
at least one processor configured to process the vibration signals and the strain signals and to derive at least one vital sign parameter of the patient based on the processed vibration signals and the processed strain signals including at least one of a patient heart performance parameter or a patient respiration performance parameter.

2. The system of claim 1, wherein the vibration sensor comprises a geophone.

3. The system of claim 1, wherein the vibration sensor comprises a piezoelectric element.

4. The system of claim 1, wherein the set of load cells is isolated from environmental vibrations by a set of isolation pads underlying the load cells.

5. The system of claim 4, wherein the set of isolation pads is formed of elastic material.

6. The system of claim 1, wherein the set of load cells is configured such that there is a load cell in every load path carrying weight of the resting patient.

7. The system of claim 1, wherein the set of load cells comprises a plurality of load cells.

8. The system of claim 1, wherein the at least one processor comprises:
analog conditioning circuitry configured to amplify and filter the vibration signals and the strain signals; and
digital conversion circuitry configured to digitize the amplified and filtered vibration and strain signals.

9. The system of claim 8, wherein the analog conditioning circuitry is configured to filter the vibration signals to isolate frequencies of interest.

10. The system of claim 9, wherein the analog conditioning circuit includes at least one of a high-pass filter, a low-pass filter, or a bandpass filter to filter the vibration signals.

11. The system of claim 9, wherein the analog conditioning circuit includes at least one of:
a Sallen-Key topology fourth-order Chebyshev high-pass filter having a 3 dB point of 10 Hz or lower;
a Sallen-Key topology fourth-order Chebyshev low-pass filter having a 3 dB point of no less than 20 Hz; or
a bandpass filter with a bandwidth of at least 10 Hz centered at 15 Hz.

12. The system of claim 9, wherein the analog conditioning circuitry is configured to amplify the filtered vibration signals by a factor of at least 200.

13. The system of claim 8, wherein the analog conditioning circuitry is configured to sum the strain signals from the set of load cells, amplify the summed strain signals, and filter the summed and amplified strain signals.

14. The system of claim 13, wherein the analog conditioning circuitry includes a resistive Wheatstone bridge to sum the strain signals.

15. The system of claim 13, wherein the analog conditioning circuitry includes at least one of:
a three op-amp instrumentation amplifier circuit for amplifying the summed strain signals; or
a low-pass filter using an RC circuit with a characteristic frequency of approximately 1 Hz for filtering the summed and amplified strain signals.

16. The system of claim 13, wherein the analog conditioning circuitry is configured to amplify the summed strain signals by a factor of at least 1000.

17. The system of claim 8, wherein the digital conversion circuitry comprises at least one analog-to-digital converter.

18. The system of claim 17, wherein the digital conversion circuitry is configured to sample the amplified and filtered vibration signals at a frequency of greater than 100 Hz.

19. The system of claim 17, wherein the digital conversion circuitry is configured to sample the amplified and filtered strain signals at a frequency of greater than 2 Hz.

20. The system of claim 8, wherein the at least one processor further comprises:
vital sign monitoring circuitry configured to derive the at least one vital sign parameter from the digitized vibration and strain signals.

21. The system of claim 20, wherein the vital sign monitoring system is configured to identify samples of digitized vibration and strain signals relating to the at least one vital sign parameter and derive the at least one vital sign parameter from the identified samples.

22. The system of claim 20, wherein:
the platform, the vibration sensor, the set of load cells, the analog conditioning circuitry, the digital conversion circuitry, and the vital sign monitoring circuitry are integrated in a single device.

23. The system of claim 20, wherein:
the platform, the vibration sensor, the set of load cells, the analog conditioning circuitry, and the digital conversion circuitry are integrated into a first device; and
the vital sign monitoring circuitry is remote from the first device and receives the digitized vibration and strain signals from the first device via a communication system.
